(19) United States
(12) Patent Application Publication
(10) Pub. No.: US 2005/0222178 A1
(43) Pub. Date: Oct. 6, 2005

(54) Process

(76) Inventors: Mark Shipton, Stevenage (GB); Neil Michael Smith, Stevenage (GB); Andrew Jonathan Whitehead, Stevenage (GB); Marian Wladyslaw Wood-Kaczmar, Stevenage (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 10/518,246

(22) PCT Filed: Jun. 16, 2003
(86) PCT No.: PCT/EP03/06412

§ 371 Date: Dec. 16, 2004

Related U.S. Application Data
(60) Provisional application No. 60/388,765, filed on Jun. 17, 2002.

(51) Int. Cl. A61K 31/52; C07D 473/14

(52) U.S. Cl. 514/263.2; 544/277

(57) ABSTRACT

The present invention relates to an improved process for the preparation of heterocyclyl substituted adenosine derivatives. More particularly the invention is concerned with preparation of particular physical forms of (2S, 3S, 4R, 5R)-2-(5 tert-butyl-[1,3,4]oxadiazol-2-yl)-5-[6-(4-chloro-2-fluoro-phenylamino)-9H-purin-9-yl]tetrahydro-furan-3, 4-diol.

The present invention relates to an improved process for the preparation of heterocyctyl substituted adenosine derivatives. More particularly the invention is concerned with preparation of particular physical forms of (2S, 3S, 4R, 5R)-2-(5 tert-butyl-[1,3,4]oxadiazol-2-yl)-5-[6-(4-chloro-2-fluoro-phenylamino)-9H-purin-9-yl]-tetrahydro-furan-3,4-diol, pharmaceutical compositions thereof and its use in therapy.

(2S, 3S, 4R, 5R)-2-(5 tert-Butyl-[1,3,4]-oxadiazol-2-yl)-5-[6-(4-chloro-2-fluoro-phenylamino)-9H-purin-9-yl]-tetrahydro-furan-3,4-diol has activity at the Adenosine A1 receptor.

WO 99/67262 (Glaxo Group Limited) discloses certain heterocyclyl adenosine derivatives including (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol, Example 14 of WO 99/67262, the structure of which is indicated below as the compound of formula (A):

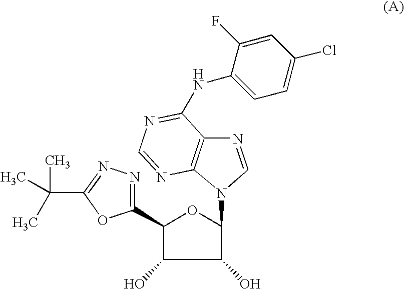

The preparation of the compound of formula (A) (hereinafter referred to as Compound A) is described in WO 99/67262. Compound A may be prepared by the reaction of 4-chloro-2-fluoroaniline with an appropriate purinyl derivative having a suitable leaving group in the 6-position of the purine ring, optionally in the presence of a solvent at elevated temperatures. Alternatively Compound A may be prepared by treating 9-{(3aR,4R,6S,6aR)-6-[5-tert-butyl-1,3,4-oxadiazol-2-yl]-2,2-dimethyltetrahydrofuro[3,4-d][1,3]dioxol-4-yl}-N-(4-chloro-2-fluorophenyl)-9H-purin-6-amine with trifluoroacetic acid followed by treatment with sodium bicarbonate. Extraction of the product into ethyl acetate followed by evaporation in vacuo provides Compound A as a buff solid.

Compound A can be obtained in polymorphic forms.

Compound A may be obtained by crystallisation under certain conditions in the form of polymorphic form I (hereinafter Polymorph I).

It has also been found that Compound A may also be crystallised in the form of polymorphic form II (hereinafter Polymorph II).

Polymorph I exhibits enhanced stability over Polymorph II at ambient temperatures, for example 15-20° C.

Polymorph II exhibits enhanced stability over Polymorph I at elevated temperatures, for example temperatures in excess of 70° C.

Polymorph I and Polymorph II may be useful in the preparation of pharmaceutical formulations.

(2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol may be prepared in polymorphic form by crystallisation of the compound under suitable conditions.

In general, (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph I may be obtained by crystallisation of the compound by heating in N,N-dimethylformamide at a temperature sufficient to effect dissolution, for example 70-90° C., and initiating crystallisation by controlled addition of water until turbidity results, and allowing to cool to ambient temperature, for example 15-25° C.

Alternatively, (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph I may be obtained by reacting 9-{(3aR,4R,6S,6aR)6-[5-tert-butyl-1,3,4-oxadiazol-2-yl]-2, 2-dimethyltetrahydrofuro[3,4-d][1,3]dioxol-4-yl}-(4-chloro-2-fluorophenyl)-9H-purin-6-amine with trifluoroacetic acid/water, neutralisation with aqueous methanolic ammonia solution, at 25-50° C. over at least one hour followed by cooling to 0-5° C.

In general, (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in the form of Polymorph II may be obtained by crystallisation of the compound by heating in methyl isobutyl ketone at reflux (117-118° C.) and allowing to cool to ambient temperature, for example 15-25°C.

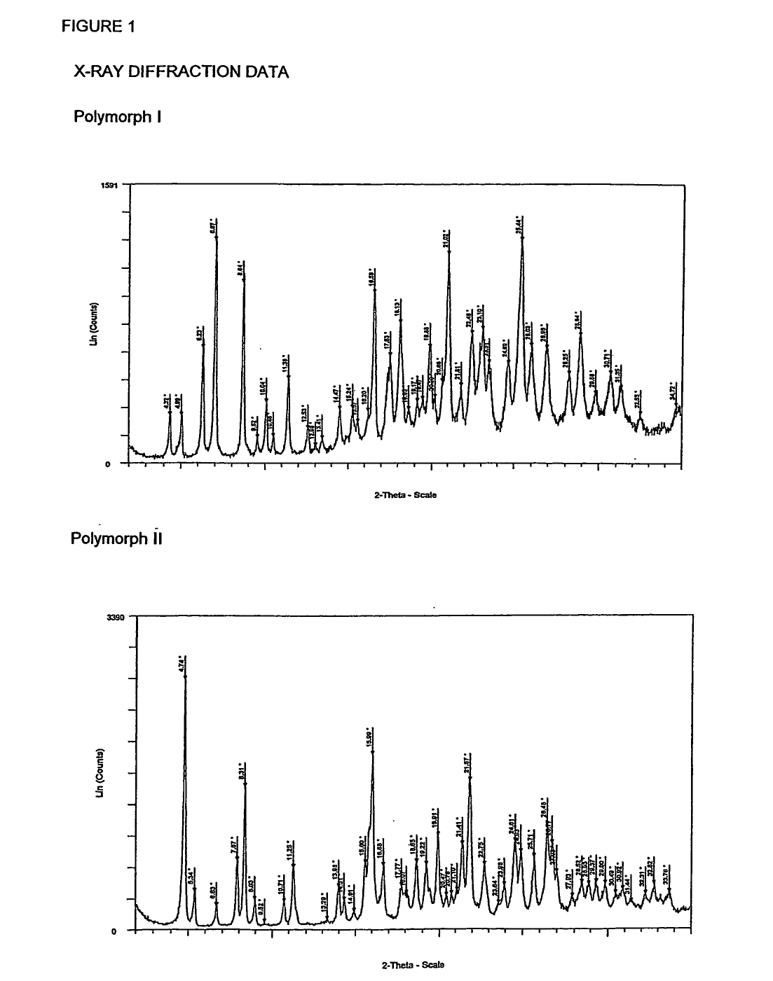

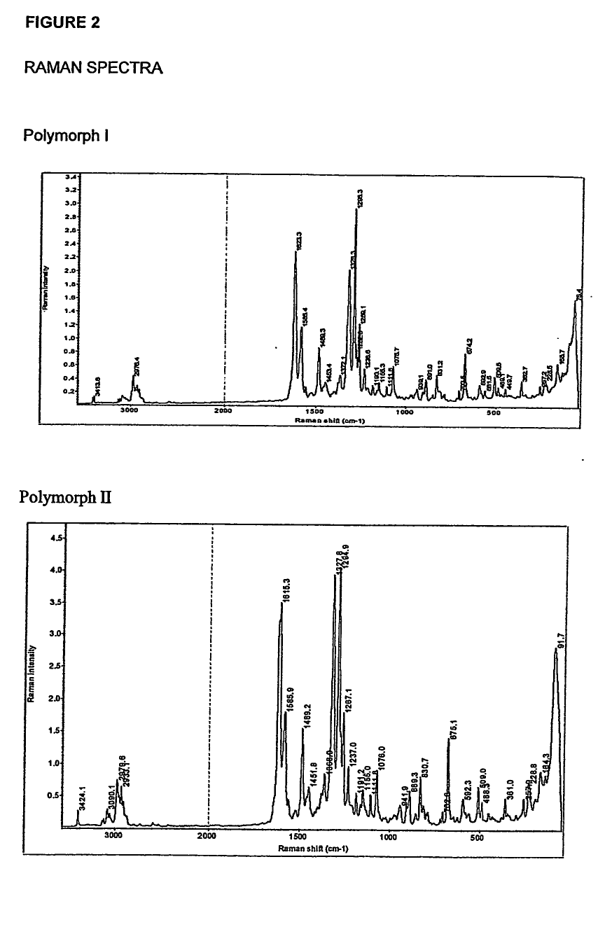

Polymorph I and Polymorph II have been characterised by X-ray powder diffraction (XRPD) studies and Raman spectroscopy as shown in FIGS. 1 and 2.

Polymorph I is characterised by having peaks in its Raman spectra at 3429, 3414 and 76 cm⁻¹, especially at 3141 and 76 cm⁻¹.

Polymorph II is characterised by having peaks in its Raman spectra at 3424, 1615 and 92 cm⁻¹.

Raman peaks are quoted to the nearest cm⁻¹.

Polymorph I is characterised by having an XRPD pattern with signals at 4.32, 4.99, 6.23, 6.97, 8.64, 10.04, 12.53, and 14.47 (degrees 2-theta).

Polymorph II is characterised by having an XRPD pattern with signals at 4.74, 5.34, 6.63, 7.87, 8.31, 8.93, 10.71, and 13.98 (degrees 2-theta).

The skilled person will recognise that XRPD peak positions are affected by differences in sample height. The peak positions quoted herein are thus subject to a variation of +/−0.15 degrees 2-theta.

When prepared using the known procedures mentioned above, the crystals of Polymorph I and Polymorph II are obtained in a highly fibrous crystal habit which makes the materials difficult to handle. Furthermore, difficulty may be experienced in controlling the polymorphic form of the isolated product.

The present invention provides a process for the preparation of Polymorph I in a crystal habit that is easy to handle, and offers control over polymorphic form.

Accordingly, in a first aspect, the present invention provides a process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph I comprising:
a) dissolving Compound A in N,N-dimethylformamide and water wherein the N,N-dimethylformamide:water ratio is in the range 2.5:1 to 1.5:1 and the dilution is at least 15 volumes; and
b) initiating crystallisation by either: adjusting the temperature to less than 25° C.; or adjusting the temperature to less than 30° C., and seeding with Polymorph I; and
c) optionally, adding toluene.

In a preferred aspect the solution of compound A is treated with decolourising charcoal prior to adjusting the temperature.

Preferably the N,N-dimethylformamide:water ratio during the crystallisation is in the range from 2.2:1 to 1.8:1, more preferably the ratio is 2:1.

Preferably the dilution of the solution prior to crystallisation of Polymorph I is 15 to 40 volumes, more preferably 15 to 30 volumes.

Preferably the solution of Compound A is cooled to 20-29° C., more preferably 25-28° C. prior to seeding.

When toluene is added during the preparation of Polymorph I, preferably it is added in an amount in the range of 1 to 5 volumes, more preferably 1 to 2 volumes.

In a preferred embodiment the present invention provides a process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph I comprising:
a) dissolving Compound A in N,N-dimethylformnamide and water wherein the N,N-dimethylformamide:water ratio is in the range 2.5:1 to 1.5:1 and the dilution is at least 15 volumes; and
b) initiating crystallisation by adjusting the temperature to less than 250° C. and seeding with Polymorph I; and
c) adding toluene.

In an alternative embodiment the present invention provides a process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph I comprising:
a) reacting 9{(3aR,4R,6S,6aR)-6-[5-tert-butyl-1,3,4-oxadiazol-2-yl]-2,2-dimethyltetrahydrofuro[3,4-d][1,3]dioxol-4-yl}N-(4-chloro-2-fluorophenyl)-9H-purin-6-amine with trifluoroacetic acid/water; and
b) neutralisation with aqueous methanolic ammonia solution at 25-50° C. over at least one hour; and
c) cooling to 0-5° C., and optionally adding toluene.

Suitable temperatures for the reaction of 9-{(3aR,4R,6S,6aR)-6-[5-tert-butyl-1,3,4-oxadiazol-2-yl]-2,2-dimethyltetrahydrofuro[3,4-d][1,3]dioxol-4-yl}-N-(4-chloro-2-fluorophenyl)-9H-purin-6-amine with trifluoroacetic acid/water include ambient temperature, e.g. 15-250° C. Suitably the TFA/water ratio is 20:1 to 5:1 v/v, preferably 12:1 to 7:1 v/v.

Suitably the neutralisation step is carried out over 1-2 hours. Preferably the reaction temperature is maintained at a temperature of 35-45 ° C. throughout the neutralisation.

Suitably the reaction mixture is cooled to 0-5° C. over at least 1 hour, preferably 1 to 2 hours.

In a preferred embodiment toluene is added to the reaction mixture.

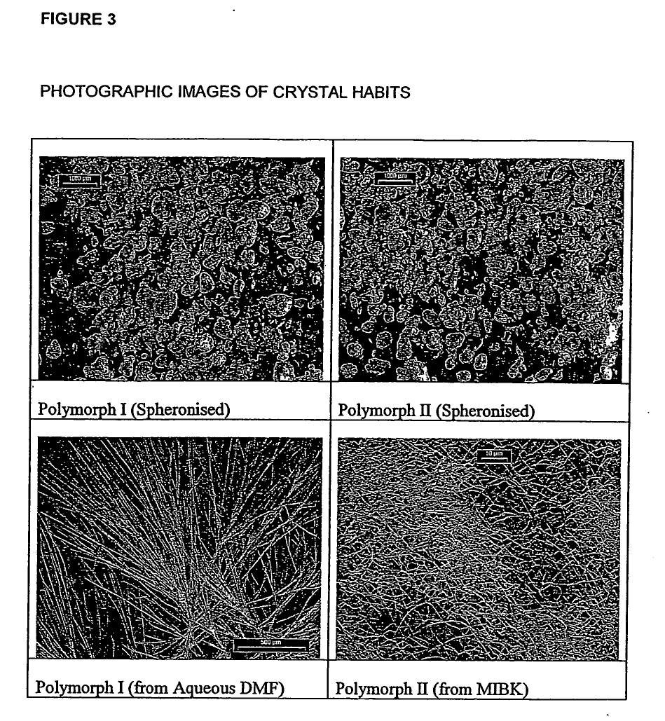

The crystal habit of Polymorph I prepared according to the present invention is of a spheronised habit as indicated in the photographic images in FIG. 3.

The present invention therefore further provides Polymorph I in spheronised habit. The invention also provides Polymorph I in spheronised habit substantially free of alternative habits.

A further aspect of the present invention is Polymorph I in a habit obtainable by a process of the present invention.

The present invention further provides a process for the preparation of Polymorph II in a crystal habit that is easy to handle, and offers control over the polymorphic form obtained.

Accordingly, the present invention additionally provides a process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph II comprising:
a) dissolving Compound A in N,N-dimethylformamide and water wherein the N,N-dimethylformamide:water ratio is in the range 2:1 to 1:2 and the dilution is at least 15 volumes; and
b) initiating crystallisation by either: adjusting the temperature to greater than 35° C.; and optionally seeding with polymorph II; and
c) optionally, adding toluene.

In a preferred aspect the solution of Compound A is treated with decolourising charcoal prior to adjusting the temperature and seeding.

Preferably the N,N-dimethylformamide:water ratio during crystallisation of Polymorph II is in the range of 1.8:1 to 1.2:1, more preferably 1.5:1 to 1.3:1, yet more preferably 1.4:1.

Preferably the dilution of the solution prior to crystallisation of Polymorph Il is 15 to 40 volumes, more preferably 15 to 30 volumes.

Preferably the temperature of the solution of Compound A is adjusted to 35-120° C., more preferably 50-70° C. yet more preferably 50-55° C., prior to crystallisation of Polymorph II.

When toluene is added during the preparation of Polymorph II, preferably it is added in an amount in the range of 1 to 5 volumes, more preferably 1 to 2 volumes.

In a preferred embodiment the present invention further provides a process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph II comprising:
a) dissolving Compound A in N,N-dimethylformamide and water wherein the N,N-dimethylformamide:water ratio is in the range 1.5:1 to 1.3:1 and the dilution is at least 15 volumes; and
b) initiating crystallisation by adjusting the temperature to greater than 35° C.; and seeding with polymorph II; and
c) adding toluene.

Preferably the N,N-dimethylformamide:water ratio during crystallisation of Polymorph II is 1.4:1.

The crystal habit of Polymorph II prepared according to the present invention is of a spheronised habit as indicated in the photographic images in FIG. 3.

The present invention therefore further provides Polymorph II in spheronised habit. The invention also provides Polymorph II in spheronised habit substantially free of alternative habits.

A further aspect of the present invention is Polymorph II in a habit obtainable by a process of the present invention.

By “substantially free” is meant containing less than 10%, preferably less than 5%, more preferably less than 2%, of alternative habits.

When used herein the dilution term “volumes” means millilitres (mL) of solvent per gram of Compound A. For example, 10 volumes means 10 mL of solvent per 1 g of Compound A.

The invention provides a pharmaceutical composition comprising polymorph I of (2S,3S,4R,5R)-2-(5-tert-butyl-[1,3,4]oxadiazol-2-yl)-S-[6-(4-chloro-2-fluorophenylamino)-9H-purin-9-yl]-tetrahydrofuran-3,4-diol in a habit obtainable by a process of the present invention and a pharmaceutically acceptable carrier and/or excipient.

This invention further provides a pharmaceutical composition comprising polymorph I of (2S,3S,4R,5R)-2-(5-tert-butyl-[1,3,4]oxadiazol-2-yl)-5-[6-(4-chloro-2-fluorophenylamino)-9H-purin-9-yl]-tetrahydrofuran-3,4-diol in spheronised habit, and a pharmaceutically acceptable carrier and/or excipient.

The invention also provides a method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph I of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in a habit obtainable by a process of the present invention.

The present invention also provides a method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph I of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in spheronised habit.

Polymorph I of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in a habit obtainable by a process of the present invention may be used for decreasing plasma free fatty acid concentration; reducing heart rate; or treating ischemic heart disease, peripheral vascular disease, stroke, pain, CNS disorder, or sleep apnoea, as described in WO 99/67262.

Polymorph I of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in spheronised habit may be used for decreasing plasma free fatty acid concentration; reducing heart rate; or treating ischemic heart disease, peripheral vascular disease, stroke, pain, CNS disorder, or sleep apnoea, as described in WO 99/67262.

Polymorph I of (2S,3S,4R,5R)-2-(5-tert-butyl-[1,3,4]-xadiazol-2-yl)-5-[6(4-chloro-2-fluorophenylamino)-9H-purin-9-yl]-tetrahydrofuran-3,4-diol in a habit obtainable by a process of the present invention may be used in the manufacture of a medicament for use in decreasing plasma free fatty acid concentration; reducing heart rate; or treating ischemic heart disease, peripheral vascular disease, stroke, pain, CNS disorder, or sleep apnoea, as described in WO 99/67262.

Polymorph I of (2S,3S,4R,5R)-2-(5-tert-butyl-[1,3,4]-oxadiazol-2-yl)5-[6-(4-chloro-2-fluorophenylamino)-9H-purin-9-yl]-tetrahydrofuran-3,4-diol in spheronised habit may be used in the manufacture of a medicament for use in decreasing plasma free fatty acid concentration; reducing heart rate; or treating ischemic heart disease, peripheral vascular disease, stroke, pain, CNS disorder, or sleep apnoea, as described in WO 99/67262.

This invention further provides for a pharmaceutical composition comprising Polymorph II of (2S,3S,4R,5R)-2-(5-tert-butyl-[1,3,4]oxadiazol-2-yl)-5-[6-(4-chloro-2-fluorophenylamino)-9H-purin-9-yl]-tetrahydrofuran-3,4-diol in a habit obtainable by a process of the present invention, and a pharmaceutically acceptable carrier and/or excipient.

This invention further provides for a pharmaceutical composition comprising Polymorph II of (2S,3S,4R,5R)-2-(5-tert-butyl-[1,3,4]oxadiazol-2-yl)-5-[6(4-chloro-2-fluorophenylamino)-9H-purin-9-yl]-tetrahydrofuran-3,4-diol in spheronised habit, and a pharmaceutically acceptable carrier and/or excipient.

The invention also provides a method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph II of (2S,3S,4R,5R)-2-(5-tert-butyl-[1,3,4]-oxadiazol-2-yl)-5-[6(4-chloro-2-fluorophenylamino)-9H-purin-9-yl]-tetrahydrofuran-3,4-diol in a habit obtainable by a process of the present invention.

The invention also provides a method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph II of (2S,3S,4R,5R)-2-(5tert-butyl-[1,3,4]-oxadiazol-2-yl)-5-[6-(4-chloro-2-fluorophenylamino)-9H-purin-9-yl]-tetrahydrofuran-3,4-diol in spheronised habit.

Polymorph II of (2S,3S,4R,5R)-2-(5tert-butyl-[1,3,4]-oxadiazol-2-yl)-5-[6-(4-chloro2-fluorophenylamino)-9H-purin-9-yl]-tetrahydrofuran-3,4-diol in a habit obtainable by a process of the present invention may be used for decreasing plasma free fatty acid concentration; reducing heart rate; or treating ischemic heart disease, peripheral vascular disease, stroke, pain, CNS disorder, or sleep apnoea, as described in WO 99/67262.

Polymorph II of (2S,3S,4R,5R)-2-(5tert-butyl-[1,3,4]-oxadiazol-2-yl)-5-[6-(4-chloro-2-fluorophenylamino)-9H-purin-9-yl]-tetrahydrofuran-3,4-diol in spheronised habit may be used for decreasing plasma free fatty acid concentration; reducing heart rate; or treating ischemic heart disease, peripheral vascular disease, stroke, pain, CNS disorder, or sleep apnoea, as described in WO 99/67262.

Polymorph II of (2S,3S,4R,5R)-2-(5tert-butyl-[1,3,4]-xadiazol-2-yl)-5-[6-(4-chloro-2-fluorophenylamino)-9H-purin-9-yl]-tetrahydrofuran-3,4-diol in a habit obtainable by a process of the present invention may be used in the manufacture of a medicament for use in decreasing plasma free fatty acid concentration; reducing heart rate; or treating ischemic heart disease, peripheral vascular disease, stroke, pain, CNS disorder, or sleep apnoea, as described in WO 99/67262.

Polymorph II of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in spheronised habit may be used in the manufacture of a medicament for use in decreasing plasma free fatty acid concentration; reducing heart rate; or treating ischemic heart disease, peripheral vascular disease, stroke, pain, CNS disorder, or sleep apnoea, as described in WO 99/67262.

Suitable pharmaceutically acceptable carriers and excipients are described in WO 99/967262.

WO 99/67262 (Glaxo Group Limited) is incorporated by reference herein as though fully set forth.

The following examples illustrate the invention but are not intended as a limitation thereof.

The following examples illustrate aspects of this invention but should not be construed as limiting the scope of the invention in any way.

### EXAMPLES

Unless otherwise indicated, (2S,3S,4R,5R)-2-(5-tert-Butyl-[1, 3,4]oxadiazol-2-yl)-5-[6-(4-chloro-2-fluorophenylamino)-9H-purin-9-yl]-tetrahydrofuran-3,4-diol (Compound A) was prepared according to the methods described in WO 99/67262.

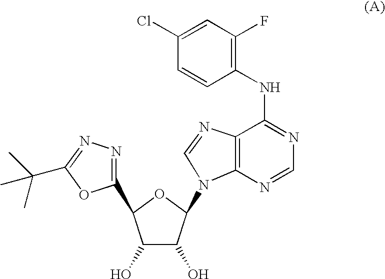

### Example 1

### (2S, 3S, 4R, 5R)-2-5 tert-Butyl-[1,3,4]oxadiazol-2-yl)-5-[6-(4-chloro-2-fluoro-Phenylamino)-9H-purin-9-yl]-tetrahydro-furan-3,4-diol (Compound A) Polymorphic Form I

Compound A (337 g) was dissolved in a warm mixture of N,N-dimethylformamide (DMF, 3.37 L) and water (1.12 L). Decolourising charcoal (84.2 g) was added and the suspension stirred at 60° C. for 1 hour. The charcoal was removed by filtration and the filter washed with a mixture of DMF (1.12 L) and water (374 mL). The combined filtrate and wash was then cooled to 25-28° C. Water (740 mL) was added and the solution seeded with Compound A (Polymorph I). The resulting suspension was then stirred overnight at 20-25° C. Water (2.25 L) was added and the suspension stirred for 2 hours. Toluene (472 mL) was added and the product collected by filtration, washed with 1:1 aqueous DMF (840 mL) and water (8.4 L), and then dried in vacuo at 40-45° C. to give Compound A (Polymorph I) as an off white solid (277.5 g, 82% recovery).

### Example 2

### (2S, 3S, 4R, 5R)-2-(5 tert-Butyl-[1,3,4]oxadiazol-2-yl)-5-[6-(4chloro-2-fluoro-phenylamino)-9H-purin-9-yl]-tetrahydro-furan-3,4-diol (Compound A) Polymorphic Form II

Compound A (20 g) was dissolved in a warm mixture of N,N-dimethylformamide (DMF, 200 mL) and water (100 mL). Decolourising charcoal (5.0 g) was added and the suspension stirred at 60° C. for 1 hour. The charcoal was removed by filtration and the filter washed with a mixture of DMF (66.6 mL) and water (33.3 mL). The temperature of the combined filtrate and wash was adjusted to 50-55° C., water (53.4 mL) was added and the solution seeded with Compound A (Polymorph II). The resulting suspension was then stirred overnight at 50-55° C. Water (80 mL) was added and the suspension stirred for 2 hours. The mixture was then cooled to 20-25° C. and stirred at this temperature for 1 hour. Toluene (28 mL) was added and the product collected by filtration, washed with 1:1 aqueous DMF (50 mL) and water (500 mL), and then dried in vacuo at 40-45° C. to give Compound A (Polymorph II) as an off white solid (17.0 g, 85% recovery).

### Example 3

### (2S, 3S, 4R, 5R)-2-(5 tert-Butyl-[1,3,4]oxadiazol-2-yl)-5[6-(4-chloro-2-fluoro-Phenylamino)-9H-purin-9-yl]-tetrahydro-furan-3,4-diol (Compound A) Polymorphic Form I (alternative process)

9-{(3aR,4R,6S,6aR)-6-[5-tert-butyl-1,3,4-oxadiazol-2-yl]-2,2-dimethyltetrahydrofuro[3,4-d][1,3]dioxol-4-ly}-N-(4-chloro-2-fluorophenyl)-9H-purin-6-amine (Intermediate X) may be prepared according to the methods described in WO 99/67262 (Intermediate 63, page 122).

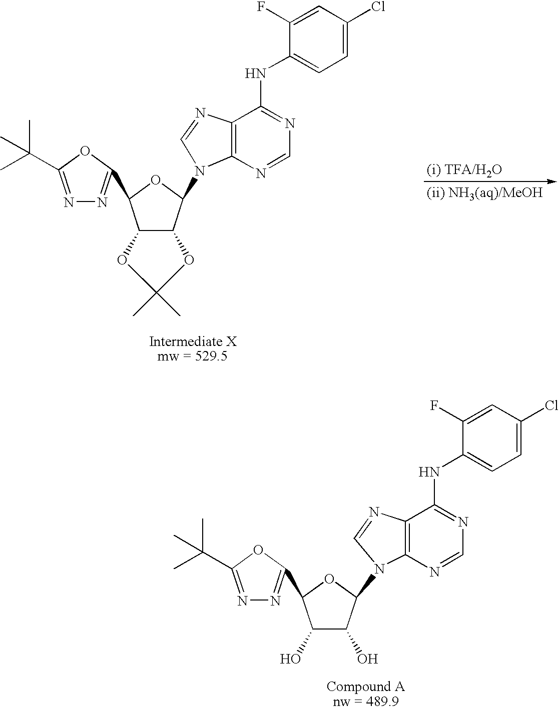

9-{(3aR,4R,6S,6aR)-6-[5-tert-butyl-1,3,4-oxadiazol-2-yl]-2,2-dimethyltetrahydrofuro[3,4-d][1,3]dioxol-4-yl}N-(4-chloro-2-fluorophenyl)-9H-purin-6-amine (Intermediate X, 10 g) was added portion wise to 9:1 v/v TFA/water (38 ml) at 20° C. and the mixture stirred for 3-6 hours at 20-25° C. The reaction mixture is then neutralised by addition over a 1-1.5 h period to a solution of 880 ammonia (28 ml), water (85 ml) and methanol (127 ml) at 35-45° C. The mixture is then cooled to 0-5° C. over a 1 h period and stirred for a further 0.5 h at 0-5° C.*. The product is collected by filtration, washed with water (3×50 ml) and dried under vacuum at 50° C. to constant weight to give the product as a white solid (8.42 g, 90.9% yield).
* Toluene (10 ml) can be added at this point to give a faster filtering product

X-Ray Powder Diffraction

The sample preparation and acquisition conditions were as follows:

Samples were lightly ground and packed into silicon cup with a 12 mm (diameter)×0.5 mm cavity. Data were acquired using a Bruker D8 Advance X-Ray diffractometer configured with a Cu anode, primary and secondary Soller slits, secondary monochromator and scintillation counter. The generator was operated at 40 kV 40 mA. Variable divergence and antiscatter slits were set at 12 mm irradiated area, and the detector slit was set at 0.1 mm. A locked coupled step scan with 0.02 degrees 2 -theta step was used. The sample was rotated.

Data obtained for Polymorph I and Polymorph II are shown in Figure I.

Raman Spectroscopy

Raman spectra were acquired using a Nicolet 960 ESP FT-Raman spectrometer. Samples were held in glass vials; spectra of 5 different points on a sample were averaged. Data collection parameters include: Laser power: 400 mW, Resolution: 4 cm-¹, Sample gain: 1.0, Detector. InGaAs, Beamsplitter: CaF2, Correction: none, Zero filling: none, Apodization: Happ-Genzel, Phase correction: Power spectrum.

Raman spectra of Polymorph I and Polymorph II are shown in FIG. 2.

Photographic image of polymorph I and Polymorph II in spheronised habit are shown in FIG. 3. Images of Polymorphs I and II in the fibrous habit obtained using known procedures are shown by way of comparison.

1-15. (canceled)

16. A process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph I comprising:
a) reacting 9-{(3aR,4R,6S,6aR)-6-[5-tert-butyl-1,3,4-oxadiazol-2-yl]-2,2-dimethyltetrahydrofuro[3,4-d][1,3]dioxol-4-yl}-N-(4-chloro-2-fluorophenyl)-9H-purin-6-amine with trifluoroacetic acid/water;
b) neutralizing with aqueous methanolic ammonia solution at 25-50° C. over at least one hour; and
c) cooling to 0-5° C., optionally adding toluene.

17. A process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph I comprising:
a) dissolving Compound A in N,N-dimethylformamide and water wherein the N,N-dimethylformamide:water ratio is in the range 2.5:1 to 1.5:1 and the dilution is at least 15 volumes;
b) initiating crystallisation by either adjusting the temperature to less than 25° C.; or adjusting the temperature to less than 30° C., and seeding with Polymorph I; and
c) optionally, adding toluene.

18. A process for preparing (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol (Compound A) as Polymorph II comprising:
a) dissolving Compound A in N,N-dimethylformamide and water wherein the N,N-dimethylformamide:water ratio is in the range 2:1 to 1:2 and the dilution is at least 15 volumes;
b) initiating crystallisation by adjusting the temperature to greater than 35° C.; and optionally seeding with polymorph II; and
c) optionally, adding toluene.

19. (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol as Polymorph I in spheronised habit.

20. (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol as Polymorph I in a habit obtainable by a process of claim 16.

21. (2S,3S,4R,5R)-2-(5-tert-Butyl-[1,3,4]-oxadiazol-2-yl )-5-[6-(4-chloro-2-fluorophenylamino)-9H-purin-9-yl]-tetrahydrofuran-3,4-diol as Polymorph I in a habit obtainable by a process of claim 17.

22. (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol as Polymorph II in spheronised habit.

23. (2S,3S,4R,5R)-2-(5-tert-Butyl-[1,3,4]-oxadiazol-2-yl)-5-[6-(4-chloro-2-fluorophenylamino)-9H-purin-9-yl]-tetrahydrofuran-3,4-diol as Polymorph II in a habit obtainable by a process of claim 18.

24. A pharmaceutical composition comprising Polymorph I in a habit according to claim 19, and a pharmaceutically acceptable carrier and/or excipient.

25. A pharmaceutical composition comprising Polymorph I in a habit according to claim 20, and a pharmaceutically acceptable carrier and/or excipient.

26. A pharmaceutical composition comprising Polymorph I in a habit according to claim 21, and a pharmaceutically acceptable carrier and/or excipient.

27. A pharmaceutical composition comprising Polymorph II in a habit according to claim 22, and a pharmaceutically acceptable carrier and/or excipient.

28. A pharmaceutical composition comprising Polymorph II in a habit according to claim 23, and a pharmaceutically acceptable carrier and/or excipient.

29. A method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph I of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in a habit according to claim 19.

30. A method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph I of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in a habit according to claim 20.

31. A method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph II of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in a habit according to claim 22.

32. A method of treating a patient suffering from a condition where there is an advantage in decreasing plasma free fatty acid concentration, or reducing heart rate, or treating a patient suffering from ischemic heart disease, peripheral vascular disease, stroke, pain, CNS disorder, or sleep apnoea comprising administering a therapeutically effective amount of Polymorph II of (2S,3S,4R,5R)-2-(5-tert-butyl-[1,3,4]-oxadiazol-2-yl)-5-[6-(4-chloro-2-fluorophenylamino)-9H-purin-9-yl]-tetrahydrofuran-3,4-diol in a habit according to claim 23.
